(19) United States
(12) Patent Application Publication
(10) Pub. No.: US 2003/0097002 A1
(43) Pub. Date: May 22, 2003

(54) Enantioselective reactions using two-component chiral phase transfer catalysts in multiphasic systems

(75) Inventors: William H. Pirkle, Champaign, IL (US); Seth E. Snyder, Urbana, IL (US); Alex Shvets, Urbana, IL (US)

Correspondence Address: SENNIGER POWERS LEAVITT AND ROEDEL, ONE METROPOLITAN SQUARE, 16TH FLOOR, ST LOUIS, MO, 63102, US

(73) Assignee: The Board of Trustees of the University of Illinois

(21) Appl. No.: 10/144,274

(22) Filed: May 13, 2002

Related U.S. Application Data
(60) Provisional application No. 60/290,323, filed on May 11, 2001.

(51) Int. Cl. C07D 453/04

(52) U.S. Cl. 546/184; 546/134; 562/401

(57) ABSTRACT

The present invention provides multiphasic systems comprising at least one chiral selector and an achiral phase transfer catalyst, wherein said system can be used to effect enantioselective reactions or asymmetric induction reactions.

### REFERENCE TO RELATED APPLICATIONS

This application claims priority from U.S. provisional application Serial No. 60/290,323, filed May 11, 2001, which is incorporated herein by reference in its entirety.

### FIELD OF THE INVENTION

This invention relates to the simultaneous use of at least one chiral selector and a phase transfer catalyst to effect enantioselective reactions or asymmetric induction reactions, both types of reactions having utility in a variety of applications.

### BACKGROUND OF THE INVENTION

Stereoisomers are molecules that differ from each other only in the way their atoms are oriented in space. The particular arrangement of atoms that characterize a particular stereoisomer is known as its absolute configuration. This configuration is typically specified by known sequencing rules as, for example, either + or − (also D or L) and/or R or S. Stereoisomers are generally classified as two types, enantiomers or diastereomers. Enantiomers are stereoisomers that are mirror images of each other that cannot be superimposed. Mirror-image stereoisomers that can be superimposed on each other are known as meso compounds. Diastereomers are stereoisomers that are not mirror images of each other. Diastereomers have different physical properties such as melting points, boiling points, solubility in a given solvent, densities, refractive indices, reactivity, etc. Diastereomers can usually be readily separated from each other by conventional methods, such as fractional distillation, fractional crystallization, or chromatography on achiral media.

Enantiomers, however, present special challenges because many of their physical properties are identical. They generally cannot be separated by conventional methods, especially if they are in the form of a racemic mixture (one which includes equal proportions of each of a pair of enantiomers). Thus, they cannot be separated by fractional distillation because their boiling points are identical and they usually cannot be separated by fractional crystallization because their solubilities are identical. They also generally cannot be separated by conventional chromatography using achiral media because they are usually held equally onto the adsorbent.

Accordingly, the development of catalytic systems which cause the selective reaction of one enantiomer of a racemate or which effect an asymmetric induction reaction on an achiral starting material have been sought for many years. Even today, the development of such catalysts is being actively pursued by many research groups. Present catalytic systems are primarily limited to the use of either certain transition metals or enzymes. Use of purely synthetic organic materials as chiral phase transfer catalysts has met with limited success. For example, some asymmetric syntheses have been achieved by the use of one-component chiral phase transfer catalysts (CPTC) primarily derived from cinchona alkaloids. See, e.g., Corey, et al., J. Am. Chem. Soc., 1997, 119, 12414-12415. In general, existing catalytic systems are narrowly circumscribed in the reactions for which they can be effectively utilized.

### SUMMARY OF THE INVENTION

Among the several objects of the invention, therefore, may be noted the provision of methods for obtaining a variety of organic compounds in substantial enantiomeric purity. These methods facilitate and expand the scope of enantioselective reactions promoted by phase transfer catalysts. Further, catalytic systems are provided which combine enantioselective transport with chemical reaction, a process that is effective in the kinetic resolution of certain racemates.

Briefly, therefore, one aspect of the present invention is a method for enantioselective reaction of one enantiomer of a racemate (or near racemate) in a biphasic solvent system. The method comprises forming a reaction mixture that includes a pair of enantiomers, a chiral selector, an achiral phase transfer catalyst and aqueous and organic solvents. The term chiral selector is used to mean a highly enantioenriched organic compound, largely or totally synthetic, which complexes either an achiral starting material or preferentially complexes one enantiomer of a racemic (or near racemic) starting material or an intermediate derived from the starting material. In one example of this embodiment, the chiral selector enantioselectively complexes one enantiomer of a racemic mixture of ion pairs formed from the racemic starting material and the achiral phase transfer catalyst. This results in the preferential transport of the complexed ion pair from a first liquid phase into a second liquid phase which contains an achiral reagent which then reacts with the transported, preferentially complexed, ion pair. While the two phases will often be immiscible liquids, the multiphasic system may be comprised of a liquid phase and a solid phase or two immiscible liquid phases, one of which may be saturated with and which may contain undissolved solid. After reaction of the transported enantiomer, its separation from the unreacted second enantiomer may be achieved by conventional means.

The present invention is further directed to a novel method for converting a racemic (or near racemic) substance into an enantiomerically enriched product in a multiphasic solvent system. The method comprises forming a reaction solution that includes the racemic (or nonracemic) starting material, a chiral selector, a phase transfer catalyst, aqueous and organic solvents and an achiral basic and/or nucleophilic reagent, possibly, but not necessarily, an anion. In this system, the chiral selector enantioselectively complexes with a first enantiomer of the pair of enantiomers, thus altering its reactivity toward the basic/nucleophilic reagent affiliated with the phase transfer catalyst. The subsequent reaction occurs in a chiral environment and results in an enantioselective reaction. A variation of this theme would entail the use of an ion-pair formed from an achiral substrate and a phase transfer catalyst that reacts with an achiral reagent while the ion-pair is complexed with the selector. This reaction results in an asymmetric induction.

Further provided are mixtures of chiral selectors, wherein the selectors comprised in said mixtures comprise an identical “core” structure that is responsible for chiral recognition and at least one different substituent attached to said core. These different substituents do not negatively affect enantioselectivity but rather contribute to the different sizes and shapes of said chiral selectors. By using a mixture of chiral selectors in a solution, the solubility of the selectors is increased, thereby allowing for increased enantioeselectivity. Preferably, the chiral selector mixtures comprise proline-derived chiral selectors. In another preferred embodiment, chiral selectors comprise (S)-N-butanoylproline-3,5-dimethylanilide (BPA) and (S)-N-pivaloylproline-3,5-dimethylanilide (PPA).

The mixtures of chiral selectors find application in all of the above-mentioned methods. Furthermore, said compositions can be utilized in membrane systems that are used in separation of enantiomers.

Other objects and features will be in part apparent and in part pointed out hereinafter.

### BRIEF DESCRIPTION OF THE FIGURES

FIG. 1 is a graphic representation of partitioning of DNB-leucine utilizing tetrahexyl ammonium chloride (THAC) as phase transfer catalyst and (S)-N-butanoylproline-3,5-dimethylanilide (BPA) and (S)-N-pivaloylproline-3,5-dimethylanilide (PPA) as chiral selectors.

FIG. 2 is a graphic representation of partitioning of DNB-leucine utilizing tetrahexyl ammonium bromide (THAB) as phase transfer catalyst and (S)-N-butanoylproline-3,5-dimethylanilide (BPA) and (S)-N-pivaloylproline-3,5-dimethylanilide (PPA) as chiral selectors.

### ABBREVIATIONS AND DEFINITIONS

To facilitate understanding of the invention, a number of terms are defined below:

“Absolute configuration” refers to the actual three-dimensional structure of a chiral molecule. Absolute configurations are specified verbally by the Cahn-Ingold-Prelog R,S convention and are represented on paper by Fisher projections.

“Acyl” refers to an organic acid group in which the OH of the carboxyl group is replaced by some other substituent.

“Acylation” refers to the introduction of an acyl group into a molecule.

“Alkyl” refers to a straight-chain, branched or cyclic saturated aliphatic hydrocarbon, having one to about twenty carbon atoms or, preferably, one to about twelve carbon atoms. Typical alkyl groups include methyl, ethyl, propyl, isopropyl, butyl, isobutyl, tertiary butyl, pentyl, hexyl and the like.

“Alkenyl” embraces linear or branched radicals having at least one carbon-carbon double bond of two to about twenty carbon atoms or, preferably, two to about twelve carbon atoms.

“Alkynyl” embraces linear or branched radicals having at least one carbon-carbon triple bond of two to about twenty carbon atoms or, preferably, two to about twelve carbon atoms.

“Aryl” refers to an aromatic group which has at least one ring having a conjugated pi electron system and includes carbocyclic aryl, heterocyclic aryl and biaryl groups.

“Amide” refers to —C(O)—NH—R, where R is alkyl, aryl, alkylaryl or hydrogen.

“Carboxy” or “carboxyl” denote —CO₂H.

“Carboxyalkyl” denotes —CO₂R.

“Chiral”, as used herein, refers to having handidness. Chiral molecules do not have a plane of symmetry and are not superimposable on their mirror images. “Achiral” molecules are opposite of chiral molecules, i.e. they have a plane of symmetry and are thus superimposable on their respective mirror images.

“Chiral complexing agent” as used herein refers to a chiral reagent of significant enantiomeric purity, which is capable of transiently interacting with a significantly greater affinity with one enantiomer of a target compound than it does with the other enantiomer of the same target compound.

“Chiral complexing agent” is also referred to herein as “chiral selector.”

“Enantiomers” as used herein, refer to stereoisomers of a chiral substance that are mirror images of each other.

“Ester” denotes an organic compound or radical which may be derived from acids by the exchange of the replaceable hydrogen of the latter for an organic radical.

“Halo” or “halogeno” means halogens such as fluorine, chlorine, bromine or iodine, or the radicals thereof.

“Hydrophilic” or “lipophobic” refers to a “water-loving” affinity, i.e. having the ability to dissolve in water and/or water-comprising solutions.

“Lipophilic” or “hydrophobic” refers to a “lipid-loving” affinity, i.e. having the ability to dissolve in organic solvents.

“Racemic mixture”, as used herein, refers to an optically inactive or equimolar mixture of two enantiomers, usually produced as a result of a chemical reaction at a chiral center where neither enantiomeric product is preferred.

“Resolving enantiomers” or “resolution” refers to a process of separating pairs of enantiomers from an enantiomeric mixture.

“Water-immiscible organic solvent” refers to an organic solvent which has a maximum solubility in water of 10% at 25° C. and forms non-homogenous solutions with water. The organic solvent concentration is expressed as percentage (%) (volume/volume) and is based on the volume of the entire non-homogenous system, which includes both the aqueous and organic components.

### DESCRIPTION OF THE PREFERRED EMBODIMENT

In accordance with the present invention, it has been discovered that a kinetic resolution of an enantiomeric mixture may be achieved by the use of a chiral complexing agent (sometimes referred to herein as a chiral selector) in a reaction mediated by a phase transfer catalyst. In general, the kinetic resolution is carried out in a multiphase system which comprises an aqueous phase, an organic solvent phase, a phase transfer catalyst, an enantiomeric mixture initially present in the aqueous phase or the organic solvent phase, and a species which is reactive with at least one member of the enantiomeric mixture. The chiral complexing agent preferentially associates with one member of the enantiomeric mixture, thereby influencing the rate of accumulation of reaction product derived from that member of the enantiomeric mixture; that is, the rate of accumulation of reaction product derived from the enantiomer which preferentially associates with the chiral selector, depending on the reaction used to separate enantiomers, will be initially significantly greater than or less than the rate of accumulation of reaction product derived from the other member of the enantiomeric mixture.

Stated another way, in accordance with the present invention, two-component chiral phase transfer catalysts comprising a chiral selector and a phase transfer catalyst (PTC), such as an achiral phase transfer catalyst (APTC), are capable of effecting enantioselective reactions in biphasic solvent systems. In general, the use of chiral complexing agents (chiral selectors) permits or enhances the enantioselectivity of reactions catalyzed by phase transfer catalysts. This greatly extends the scope of enantioselective reactions promoted by phase transfer catalysts. Further, relative to a one-component chiral phase catalyst, the two-component chiral phase transfer catalyst permits the use of a variety of different chiral selectors with a given APTC or, alternatively, a variety of different APTCs with a given chiral selector.

The system described herein is at least biphasic, i.e., it contains an aqueous phase and a water-immiscible phase, such as an organic solvent phase, which share a liquid-liquid interface. An enantiomeric mixture, such as a racemic mixture or a near-racemic mixture to be resolved, can be dissolved in either the aqueous or organic solvent phase, depending on the phase transfer catalyst mediated reaction that is used to kinetically resolve the enantiomers. For example, in embodiments in which an esterification reaction is used to resolve a racemic mixture, said mixture may be dissolved in the aqueous phase. Alternatively, in embodiments in which hydrolysis reactions or reactions that involve formation of Meisenheimer complexes as described herein are used to resolve a racemic mixture, the mixture may be dissolved in the organic phase. In one embodiment, the racemate to be resolved is a racemic mixture of an N-acylated amino acid. In a preferred embodiment, the racemic mixture is a racemate of a DNB-derived amino acid, such as, for example, the racemate of DNB-leucine as illustrated herein.

The phase transfer catalyst used in the system of the present invention may be essentially any phase transfer catalyst which shuttles an anion (an enantiomer or a reactive species) from the aqueous phase to the organic phase in which a reaction occurs. Exemplary phase transfer catalysts include quaternary ammonium salts, quaternary phosphonium salts, quaternary guanadinium salts and N-alkyl-4-dialkylaminopyridinium salts. The use of phase transfer catalysts offers several advantages: 1) a properly chosen phase transfer catalyst can extract almost any anion into almost any organic medium, thus allowing for its applicability in a wide variety of reactions, 2) phase transfer catalysts enable relatively greater selectivity since phase transfer catalysts lower the energy of activation, thus allowing for reduction in times and temperatures of reactions, 3) phase transfer catalysts allow for the use of less expensive and hazardous bases (e.g. hydroxide is easily transferred and activated in a number of organic phases). In one embodiment of the present invention, the phase transfer catalyst is an achiral phase transfer catalyst such as, for example, a quaternary ammonium salt (also referred to herein as “quat”). In another embodiment, the quaternary ammonium salt comprises tetra n-hexyl ammonium halide. For example, the tetra n-hexyl ammonium halide may be tetra n-hexyl ammonium chloride (THAC) or tetra n-hexyl ammonium bromide (THAB).

Chiral selectors were initially developed for the chromatographic separation of enantiomers. Several chiral selectors that were designed for use in chiral stationary phases have been shown to effect first-order asymmetric transfer in non-polar solvents (see, e.g., Pirkle, W. H. and Reno, D. S., J. Am. Chem. Soc, 1987, 109:7189, and Pirkle, W. H., Murray, P. G., J. Chromatogr. A., 719:299-305, 1996). In general, chiral selectors are totally or largely synthetic compounds which exhibit a significantly greater affinity for one enantiomer of a target compound than they do for the other enantiomer. Chiral selectors are frequently crystalline and show limited solubility in certain organic solvents such as hydrocarbons or halogenated hydrocarbons. Typically, chiral selectors show greatest enantioselective complexing ability in relatively non-polar organic solvents. Exemplary chiral selectors that may be used in methods of the present invention include but are not restricted to crown ethers, carbohydrate polymer chiral selectors (such as cellulose and cellulose derivatives and amylose and amylose derivatives), modified alkaloids (such as cinchona and quinine), amides, macrocyclic antibiotics (such as ansamacrol ides, macrolides, macrocyclic peptides, polyenes and derivatives thereof), and α-arylalkyl amine family members (such as Whelk-O selector). In one embodiment, the chiral complexing agent used herein is a proline-derived chiral selector. In one preferred embodiment, the proline-derived chiral selector comprises N-acylated L-proline anilide (S)-2. In another embodiment, the chiral selector is hydrophobic, thus remaining in the organic solvent phase throughout the reaction.

Chiral selectors are characterized by their ability to exhibit a significantly higher affinity for one enantiomer of a target compound than they do for the other. The chiral selectors contain a “core” structure responsible for the chiral recognition of a particular enantiomer and additional “innocuous” substituents that do not affect enantioselectivity but contribute to the size and shape of the selectors. It is frequently possible to change structural features other than the core of the selector without significantly affecting the chiral recognition ability of that selector while in solution. As mentioned previously, this can be significant in certain embodiments since the selectors are frequently crystalline in nature and show limited solubility in organic solvents such as hydrocarbons or halogenated hydrocarbons.

Two factors influence the solubility of a crystalline substance, specifically the energetics of the forces holding the crystal lattice together and energetics of salvation of a dissolved selector molecule. By weakening the forces in the crystal lattice, one can increase the solubility of the selector in a given solvent. In one embodiment, therefore, the system may comprise more than one chiral selector. In this embodiment, each of the selectors preferably has the same absolute configuration and core structure but differs in at least one substituent (also referred to herein as “innocuous” group) that is attached to the core. The difference in innocuous groups contributes to the difference in size and shape among the selectors. This further tends to cause irregularities and dislocations in the lattice comprising said selectors, and may, in turn, weaken the lattice forces. Accordingly, by using a mixture of chiral selectors comprising at least two separate chiral selectors with identical cores and different “innocuous” groups attached to the core, the solubility of the mixture is increased relative to any single pure component of the mixture. Preferably, the selectors comprised in such mixtures are proline-derived. In another preferred embodiment, two proline-derived chiral selectors that may be used together comprise (S)-N-butanoylproline-3,5-dimethylanilide (BPA) and (S)-N-pivaloylproline-3,5-dimethylanilide (PPA). In another preferred embodiment, a nonpolar solvent such as a hydrocarbon or hydrocarbon mixture is used to dissolve the selector mixture since said solvent affords the strong and selective complexation as a result of the poor solvating properties of hydrocarbon solvents.

Typically, the “innocuous” groups include but are not restricted to alkyl groups of differing lengths and degrees of branching. While the energetics of salvation can be affected by these groups, the magnitude of these changes is small relative to disruptive effects to the lattice. The number of chiral selectors in a mixture of chiral selectors can be determined on a case by case basis by routine experimentation. Furthermore, the mixture of chiral selectors can include selectors of relatively low molecular weight (less than a thousand) but can also include synthetic selectors which are polymeric in nature and which can comprise a mixture of oligomers, structural isomers, or otherwise similar compounds which confer enhanced solubility to increase either the rate or enantioselectivity of the reactions.

Separation of enantiomers is further dependent on the presence of a reactive species (also referred to herein as a “pre-selected compound” or “pre-selected reagent”). The reactive species reacts with the enantiomers in the organic phase. Depending upon the type of reaction being mediated by phase transfer catalysis, either the reactive species or the enantiomers are shuttled from the aqueous phase to the organic phase by the phase transfer catalyst. Advantageously, the chiral selector influences the rate of accumulation of reaction product derived from one of the members of the enantiomeric pair. For example, if the phase transfer catalyst is shuttling the enantiomers from the aqueous phase to the organic phase, the chiral selector may cause one of the enantiomers, at least initially, to be preferentially transported relative to the other enantiomer thereby leading to a more rapid rate of accumulation of reaction product derived from the preferentially transported enantiomer in the organic phase. Alternatively, if the enantiomeric mixture is present in the organic phase and the phase transfer catalyst is shuttling a reactant from the aqueous phase to the organic phase, the preferential association between the chiral selector and one member of the enantiomeric pair can influence the relative rates of accumulation of reaction products derived from the two enantiomers.

In one embodiment, a racemic or other enantiomeric mixture to be resolved is dissolved in an aqueous layer, preferably a buffered aqueous layer, and the phase transfer catalyst, chiral selector, and reactive species (such as an esterification agent) are dissolved in an organic layer. The phase transfer catalyst then migrates into the aqueous layer, where it forms an ion pair with each enantiomer. The generation of ion pairs is a result of an interaction between an anionic site of the racemate and the phase transfer catalyst cation. The ion pair is not as strongly solvated as the racemate, thus making it more lipophilic. Significantly, the chiral selector, dissolved in the organic solvent layer, influences the relative rates of extraction of the ion pairs into the organic phase. That is, the chiral selector enhances the rate of extraction of the ion pair of the phase transfer catalyst and one enantiomer of the enantiomeric mixture relative to the ion pair of the phase transfer catalyst and the other enantiomer of the enantiomeric mixture. Once in the organic phase, the enantiomers react with a reactive species present in the organic layer, thus releasing the phase transfer catalyst and/or chiral selector for future reactions.

In another embodiment, a racemic ester to be resolved is dissolved in a non-polar organic solvent together with a chiral selector. Upon stirring the organic phase with an aqueous phase comprising a base and PTC, the more complexed enantiomer stays in the organic phase, whereas it is believed that the less complexed enantiomer is hydrolyzed to a carboxylic acid at the interface of the organic and aqueous phases. It is further believed that the majority of the carboxylic acid then crosses into the aqueous phase, thereby allowing for effective separation of the enantiomers.

Similarly, in reactions involving formation of Meisenheimer complexes (MCs), the less complexed enantiomer from the electron-deficient aromatic compound, such as racemic amide forms the MC complex. The MC complex then separates as a burgundy colored sludge-like salt, which can then be treated in such manner as to result in regeneration of the amide enantiomer having the opposite configuration of the more-complexed enantiomer that remains in the organic phase.

In general, any reaction conducted with a phase transfer catalyst can be envisioned as being conducted in the presence of a chiral selector so as to influence the stereochemical course of the reaction in accordance with the present invention. Such reactions include by way of example, dehydrohalogenations; esterifications; displacement with cyanide hydroxide, hydrolysis, fluoride, thiocyanate, cyanate, iodide sulfide, sulfite, azide nitrite, or nitrate; other nucleophilic aliphatic and aromatic substitutions; oxidations; epoxidations; Michael additions; aldol condensations; Wittig Darzens condensations; carbene reactions; thiophosphorylations; reductions; carbonylations; HCl/HBr reactions; transition metal co-catalysis, Diehls-Alder reactions, and polymerizations.

A variety of factors influence the degree of separation of the enantiomers in an enantiomeric mixture. For example, the resolution being kinetic in nature will tend to become less specific as the reaction progresses and the concentration of one of the enantiomers is depleted in the reaction mixture. In one embodiment, therefore, the reaction is terminated prior to the complete extraction of enantiomers from the aqueous phase into the organic phase. Alternatively, a continuous supply of racemate may be provided to the aqueous layer.

In addition, the ion pair formed by the phase transfer catalyst and a substrate (e.g., the reactive species or the enantiomers of the mixture) more readily partition into the organic layer as the length of the alkyl groups of a phase transfer catalyst increases. Thus, the length of the alkyl substituents on phase transfer catalysts provides an easily-modifiable variable that controls partitioning of the substrate into the organic phase. As a result, the separation can be optimized to allow for minimal achiral transport and sufficient interaction between the substrate and selector.

The extent of interaction between a substrate and chiral selector is also influenced by the concentration of the chiral selector. In general, as the concentration of the chiral selector increases, the chance of partitioned substrate interacting with selector increases as well, thereby improving the enantioselectivity of the separation. Additionally, with increased concentration of the selector, the capacity of the organic phase to hold the desired substrate increases as well. Consequently, a high concentration of chiral selector is desired in the organic solvent phase.

The degree of enantioselectivity also tends to increase with the use of a nonpolar organic phase. For example, the chiral recognition for proline-based selectors used in one embodiment of the present invention is achieved through hydrogen bonding and aromatic pi cloud interactions. Accordingly, as the polarity of the organic phase increases, the level of enantioselectivity decreases due to decreased chiral recognition. Furthermore, as the polarity of the organic phase increases, the solvent begins to affect the extent of association between the substrate and selector. Thus, non-polar organic solvents including but not restricted to hexane and decane are preferably used in the methods of the present invention.

The amounts of phase transfer catalyst, chiral selector, and reactive species for each racemate (or other enantiomeric mixture) to be resolved may be readily determined through routine experimentation. In one embodiment, for each mole equivalent of the racemate, 0.035-0.2 mole equivalents of PTC, 1.0-2.5 mole equivalents of the selector, and 0.4-0.6 mole equivalents of the reactive species may be used. In a preferred embodiment, for each mole equivalent of the racemate, 0.2 mole equivalents of PTC, 2.0 mole equivalents of the selector, and 0.5 mole equivalents of the reactive species may be used. However, one of ordinary skill in the art can easily modify these amounts to determine the best ratios for each racemate and each reaction.

In an exemplary embodiment of a kinetic resolution of the present invention, racemic N-(3,5-dinitrobenzoyl) leucine (DNBleu) (1), dissolved in dilute aqueous sodium bicarbonate, an aqueous phase solvent, is exposed to p-bromophenacyl bromide (BPB), a pre-selected reactant, tetra n-hexyl ammonium chloride (THAC), an APTC, and an N-acylated L-proline anilide, (S)-2, a chiral selector, all dissolved in a nonpolar water immiscible solvent such as carbon tetrachloride, an organic solvent (Scheme 1). Acting in conjunction, the latter two reagents selectively transport one enantiomer of the leucine derivative from the aqueous phase into the organic phase where it is alkylated by the BPB to afford enantioenriched BPB ester (3) as the product that remains in the organic layer. The unreacted enantiomer of the DNBleu remains in the aqueous layer from which it may be recovered by acidification. The lipophilicity of the APTC employed may be adjusted so that, by itself, it is less likely to effect transport of the carboxylate anion unless the lipophilic chiral selector is also involved. This suppresses the background production of racemic product, arising from transport occurring independent of the chiral selector. Alternatively, this background reaction can be lessened through use of an excess of the chiral selector even if the APTC is capable of unassisted transport.

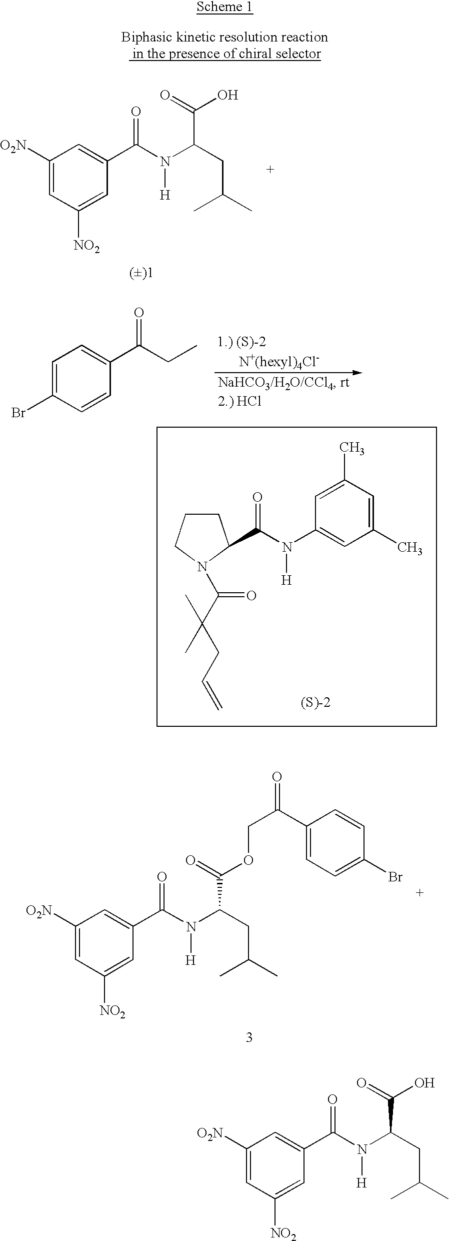

The two-component catalyst system of the present invention can be varied in a number of ways and applied to many situations. For example, in addition to the esterification reaction exemplified above, the same system may be employed using different alkylating agents (including, but not limited to: various phenacyl halides, dimethyl sulfate, methyl triflate, methyl iodide or benzyl bromide) to afford different esters of DNBleu. The DNB derivatives of other amino acids can be similarly utilized as can other anilide derivatives of proline or other anilide-like derivatives of other amino acids. One can utilize different N-acyl groups on the proline anilides or on the amino group of the anilide-like derivatives of other amino acids. Various esters or amide derivatives of N-aryl alpha amino acids complex N-3,5-dinitrobenzoyl derivatives of alpha amino acids rather enantioselectively and can be used as chiral selectors in the manner described for the proline anilide derivatives. Similarly, chiral selectors derived from pi-acidic derivatives of chiral amines, acids, amino acids, peptides, aminophosphonic acids, or alcohols may be used in manners analogous to those described herein. The efficacy and utility of different chiral selectors in enantioselective separation of racemic mixtures can be determined by routine examination.

In addition, other solvents, phase transfer catalysts, and chiral selectors may be employed other than those exemplified herein. Moreover, the two-component catalyst system may be employed to advantage for other amino acids besides N-(3,5-dinitrobenzoyl) leucine, as well as other racemic mixtures. Indeed, the N-(3,5-dinitrobenzoyl) derivatives of some synthetic amino acids prepared as the racemates do, once resolved, find usage as selectors in chiral chromatography columns capable of separating the enantiomers of many other compounds. For example, the selectors used in the commercial alpha Burke chiral HPLC column are obtained from a racemic alpha amino phosphonic acid derivative resolved chromatographically on a chiral stationary phase (CSP). This is an expensive process as presently done. Resolution of the racemic precursor by the approach described herein can be scaled up easily and should be economically advantageous. The same can be said for the selectors used in the commercial JEM 1 CSP and for any other CSP derived from the 3,5-dinitrobenzoyl derivative of a racemic alpha or beta amino acid. The present invention is not restricted to 3,5-dinitrobenzoyl derivatives, as other acylating agents can be employed. All that is required in this embodiment of the invention is that the chiral selector has a significantly greater affinity for one enantiomer of the substrate than it does for the other.

In another embodiment of the present invention, a variety of racemic esters, including but not limited to esters of α-amino acids, α-aminophosphonic acids, and α-aminolactams can be subjected to enantioselective hydrolysis. Nucleophilic basic anions may be employed in the presence of the chiral selector to effect enantioselective ester hydrolysis (Scheme 2). For example, a solution of a racemic ester of 3,5-dinitrobenzoyl leucine in a water immiscible solvent such as hexane or some other nonpolar organic solvent, if stirred with an aqueous solution of a base such as sodium hydroxide or sodium carbonate containing an achiral quaternary ammonium PTC, will undergo enantioselective hydrolysis provided an appropriate chiral selector is also present. The enantioselectivity and the rate of the reaction are affected by the type of ester, the nature and concentration of the PTC used, the organic solvent, and the temperature. In hydrolysis reactions described herein, the chiral selector protects the complexed enantiomer from hydrolysis. The course of the reaction should be monitored and, at the appropriate time, the reaction is stopped. The appropriate time to stop the reaction will be a function of the enantioselectivity of the hydrolytic process. The remaining enantioenriched ester can be recovered from the organic layer while the hydrolysis product, DNBleu, now enantioenriched in the other enantiomer, can be recovered from the aqueous layer. It is important to note that, unlike an enzymatic catalyst, the enantiomer preferentially esterified in the aforementioned embodiment is the enantiomer preferentially protected from hydrolysis. Thus, when the esterification and hydrolysis reactions are run in tandem, further enantioenrichment of the ester results.

As can be seen from Example D, the reaction rate of hydrolysis increases markedly with stirring speed. Furthermore, it has been determined that the reaction occurs at the interface of the organic and aqueous layers since the reaction rate increases with increased interfacial surface area. Hydrolysis also occurs more rapidly in the absence of the selector, demonstrating that the less complexed enantiomer is preferentially hydrolyzed.

Applicants further note that increasing the concentration of the selector slows hydrolysis and increases enantioselectivity. In addition, the presence of a phase transfer catalyst is not essential to the hydrolysis but does influence rate. While not being bound to a particular theory, it is believed that the presence of the phase transfer catalyst increases the concentration of the anionic nucleophile on the aqueous side of the interface.

Once hydrolysis has proceeded to the desired extent, isolation and acidification of the aqueous layer liberates the enantioenriched carboxylic acid. The chiral selector and the enantioenriched ester remain in the organic layer and can be recovered either by chromatography on silica or by hydrolysis of the ester and extraction of the enantioenriched acid. Therefore, it should be apparent to one skilled in the art that hydrolysis allows for separation and isolation of both enantiomers of the racemic mixture.

It should be noted that hydrolysis of the esters is faster (at least twenty times for the esters tested in Example D) in hexane than in methylene chloride and the enantioselectivity is greater as well. It is believed that this difference stems from the fact that hexane has a poorer solvating ability than methylene chloride due to a more non-polar nature, thereby resulting in increased adsorption of the ester onto the aqueous interface where hydrolysis occurs. Accordingly, organic solvents with poorer solvating ability are preferably used in hydrolysis reactions.

Due to the fact that the ester less strongly complexed with the selector is preferentially hydrolyzed and the biphasic alkylation reaction preferentially esterifies the acid enantiomer more strongly complexed, the hydrolysis scavenges the minor enantiomer produced during esterification. Thus, it is possible to run these reactions sequentially (alkylation followed by hydrolysis) to obtain each enantiomer in substantially enriched from, one as the acid, the other as the ester. Accordingly, this results in the same chemistry as seen with esterases, but with several important and beneficial differences. Compared to esterases that are also involved in enantioselective reactions, chiral selectors are inexpensive, stable, available in both enantiomeric forms, and can be recovered for reuse.

Applicants have further determined that increasing the pH of the aqueous solution once the esterification reaction is complete improves the rate of hydrolysis reaction. Such pH modifications are thus contemplated within the scope of the present invention.

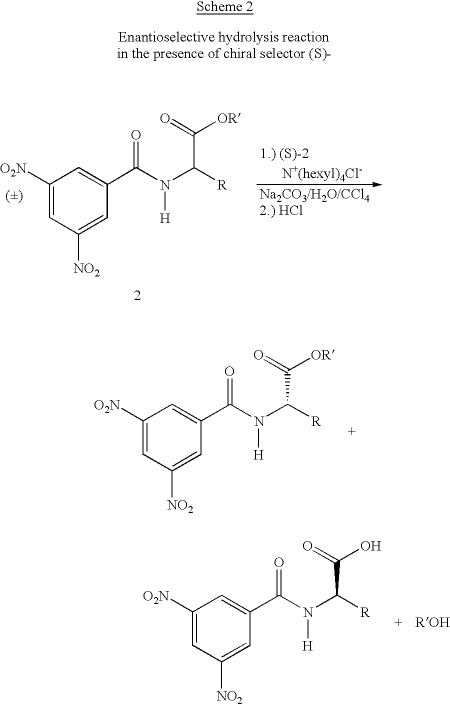

A further hydrolytic example of the invention is to effect an asymmetric induction reaction on an achiral starting material. For example, the diphenacyl ester of N-3,5-dinitrobenzoyl aminomalonic acid, dissolved in a nonpolar hydrocarbon, or other suitable solvent, if hydrolyzed in the presence of a chiral selector, aqueous base, and an achiral phase transfer catalyst, undergoes selective hydrolysis of one of the methyl ester groups, these being diastereotopic while complexed to the chiral selector and, consequently, of different reactivities. This particular reaction is complicated by the stereochemical lability of the chiral half ester and by the tendency of the half ester to be extracted into the aqueous base unless at least a half equivalent of the phase transfer catalyst is present. The latter problem can be overcome by conducting a base catalyzed transesterification reaction rather than a hydrolysis.

In yet another embodiment of the invention, the carboxyl group of a chiral acid derivative such as, but not restricted to, racemic N-3,5-dinitrobenzoyl leucine is activated with a coupling reagent such as, but not restricted to, dicyclohexylcarbodiimide and this compound is dissolved in a nonpolar hydrocarbon, or other suitable solvent, containing an appropriate chiral selector, such as (S)-2. On mixing with an aqueous solution containing an anionic nucleophile and an achiral phase transfer catalyst, transport of the anionic nucleophile into the organic layer results in enantioselective reaction owing to the different reactivities of the activated carboxyl groups of the complexed and noncomplexed enantiomers. Anionic nucleophiles such as amino acids lead to dipeptide derivatives. Mercaptide ions lead to thioesters.

In yet another embodiment of the invention, the carboxyl group of a chiral but racemic acid derivative such as, but not restricted to, N-3,5-dinitrobenzoyl leucine, is dissolved in dilute aqueous sodium bicarbonate solution containing an achiral phase transfer catalyst and stirred with a solution containing a suitable chiral selector, an activating agent such as, but not restricted to, dicyclohexyl diimide, a nucleophilic reagent such as a primary or secondary amine, a thiol, or an alcohol dissolved in a nonpolar organic solvent such as a hydrocarbon or some other suitable solvent. One enantiomer of the racemic ion pair is transported from the aqueous solution into the organic layer where it reacts with the carboxyl activating agent and then with the nucleophile to afford the enantioenriched C-terminal derivative of N-3,5-dinitrobenzoyl leucine.

The applicants have further determined that, under certain conditions, stable Meisenheimer complexes can be formed enantioselectively in the presence of a chiral selector and an achiral phase transfer catalyst using a biphasic solvent system and an appropriate nucleophile.

In addition to DNB-leu, a number of racemic amides may be utilized in two-component chiral phase transfer catalysis that result in formation of Meisenheimer complexes. It is important to note that such racemic amides should contain an electron-deficient ring system that can then be subject to nucleophilic attack. For instance, DNB derivatization result in a highly electron-deficient ring system.

Briefly, therefore, the formation of Meisenheimer complexes may be performed by dissolving the racemic amide and a suitable chiral selector in either carbon tetrachloride or hexane, and then treated with equal volume of THAC (Scheme 1). Upon addition of THAC, the organic layer becomes brightly colored. Monitoring the unreacted amide by chiral HPLC shows that the less strongly complexed enantiomer is preferentially converted to a colored Meisenheimer complex. Chiral selectors that are used should possess a high affinity for π-acidic compounds. Exemplary chiral selectors include but are not limited to S-3 and R-4, whose structures are depicted in Example E.

By way of example, a solution of a racemic amide of N-3,5-dinitrobenzoyl leucine (4) in a hydrocarbon solvent (e.g. hexane) containing a proline-derived chiral selector and half a mol equivalent of a phase transfer catalyst such as, but not limited to, THAC, will, on treatment with a 2M solution of aqueous sodium hydroxide, form a deep burgundy colored solution containing the Meisenheimer adduct (5) derived preferentially from the less complexed enantiomer of the racemic starting material (Scheme 3). The adduct has very low solubility in hexane. Upon acidic workup of the undissolved Meisenheimer adduct, one observes regeneration of 4 with considerable enantiomeric purity. The material remaining in solution is highly enriched in the other enantiomer of 4. The extent of adduct formation corresponds to the mol equivalents of PTC added, indicating that the adduct forms a stable ion pair with the PTC. The enantiomeric purity of the unreacted enantiomer is very high. This process is applicable to the resolution of the chiral selector used in the commercial Whelk-O chiral stationary phase and, as a consequence, is potentially of significance in the large-scale production of this expensive material. If the achiral PTC is immobilized, either on a solid or liquid polymer, the isolation of the new immobilized Meisenheimer adduct is facilitated using conventional means. The system is now triphasic rather than biphasic. Further examples of multiphasic systems as applicants have contemplated herein involve membrane systems used to separate enantiomers. Briefly, such membrane systems comprise three phases known as the source phase, carrier phase, and receiving phase. The membrane systems are discussed in more detail in sections below.

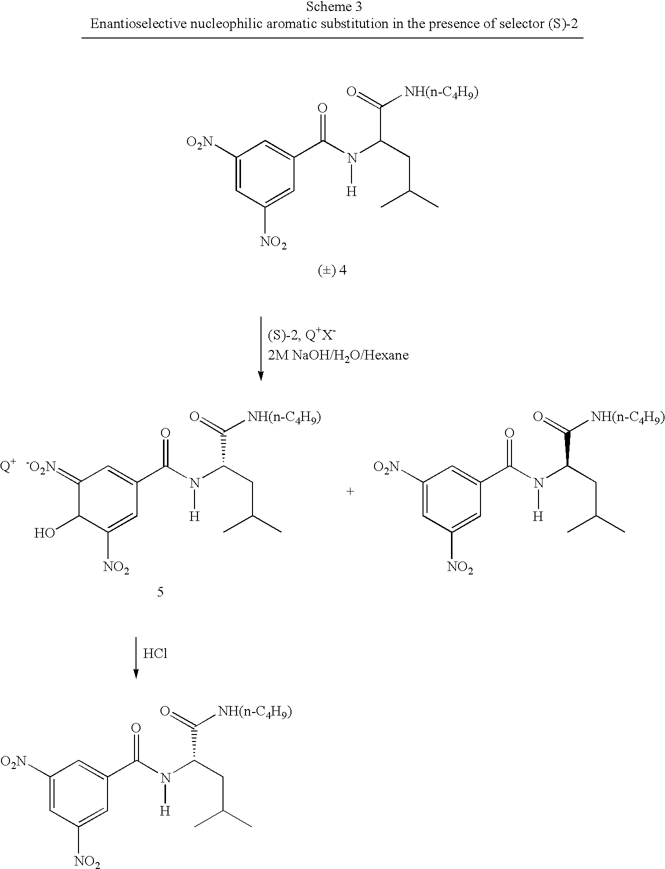

Treatment of MCs with acid leads to regeneration of amides as well as to products resulting from oxidation of MCs. The extent of oxidation is heavily dependent upon reaction times and temperature and whether oxygen has been excluded from the reaction. Reactions carried out under oxygen-free conditions give increased levels of amide regeneration upon acidification. It is believed that the small quanity of MC that is oxidized may arise through intermolecular self-oxidation by “spontaneous” aromatization (see Chupakhin et al., Nucleophilic Aromatic Substitution of Hydrogen, Academic Press, San Diego, 1994, pp.7-8). For reactions that are run in acetone-free hexane, reaction rates are increased markedly and the amount of oxidation products formed is negligible owing to phase separation of MC as a highly viscous burgundy colored sludge-like salt. Dissolution of this sludge in methylene chloride, followed by acidification, results in regeneration of the amide enantiomer having the opposite configuration of the enantiomer that remains in the hexane. In this sludge, the ratio of the quaternary ammonium cation of THAC to the anionic MC is generally greater than one, although the exact ratio is case-dependent. Consequently, loss of THAC through incorporation in the sludge-like salt gives conversions lower than those observed for CCl₄. By increasing the concentration of THAC, one increases the conversion of the amide to the MC. One of ordinary skill in the art can easily determine the amount of THAC that needs to be used in a particular reaction in order to obtain optimal conversion of the amide to the MC.

As seen with hydrolysis reactions, the enantiomer that is more strongly complexed is sequestered from the reaction. Furthermore, increased concentrations of selector give higher enantioselectivity and decreased reaction rates. The more highly associated diastereomeric complex entails π-π interaction of the electron-deficient nitroarene with the anilide portion of S-3 or the naphthyl portion of R-4. Not only does this reduce the electrophilicity of the nitroarene, but it also shields one face of the aromatic moiety from approach of the nucleophile.

Accordingly, the method can be used to resolve racemic electron-deficient aromatic compounds, some of which may be used as chiral selectors. It is also important to note that significant stereoselectivity factors were achieved even at room temperature. Furthermore, this methodology could be useful in a wide variety of enantioselective aromatic substitution reactions, including nucleophilic substitutions on heterocyclic aromatic compounds.

Applicants further note that a chiral phase transfer catalyst such as a quaternary ammonium ion may be utilized in conjunction with the chiral selector in a double asymmetric induction fashion. In some instances, this should further enhance enantioselectivity. Since both enantiomers of the applicants chiral selectors are typically available, one would utilize a chiral selector that mitigates against reaction of the enantiomer whose reaction is less favored by the chiral PTC. In other words, one would have a push-pull situation that would lead to greater enantioselectivity than could be obtained by either chiral component used separately.

In one instance a quaternary ammonium site was incorporated into a preferred chiral selector. On evaluation, this one component chiral phase transfer catalyst was found to be inferior to the corresponding two-component chiral phase transfer catalyst. This suggests that the design of a one-component PTC may be rather critical and that the two-component system has the advantage that the quaternary site is free to float spatially so that the stronger electrostatic ion pair interaction does not interfere with the simultaneous occurrence of the interactions responsible for the enantioselectivity of the selector.

Enantiomeric purity also may be adjusted by continuous supply of the racemic mixture to alleviate depletion of the more rapidly reacted enantiomer of the racemate. In general, the temperature, pH, and concentration of reactants, chiral selector or PTC of the reaction solution also may be modified as appropriate to affect the enantioselectivity, amount of product formed/enantiomeric separation provided, as well as the speed of the reaction process. These modifications should be apparent to one of ordinary skill in the art.

Although highly dependent on the particulars involved, the stereoselectivity factor (s) of reaction systems using two-component catalysts according to the invention is preferably greater than 10, more preferably greater than 20, and even more preferably greater than 30. As is well known in the art, stereoselectivity factors>10 allow for practical enantiomeric resolution. For the purposes of the present invention, the stereoselectivity factor was calculated according to the following equation:

s=In[1−C(1+ee)]/In[1−C(1−ee)],

wherein C is extent of conversion of starting material and ee is enantiomeric excess of the remaining starting material. For all chromatographic assays, conversion was determined using the selector as an internal standard. Enantiopurity is commonly reported in terms of “enantiomeric excess” (ee), which can be determined using the following formula:

% ee=(major−minor)/(major+minor)+100.

As has been disclosed herein, in order to increase the solubility of chiral selectors in organic solvents, the present invention further provides mixtures of chiral selectors that exhibit improved solubility in solvent systems.

As can be seen from Example G, different chiral selector mixtures were generated and tested in enantioselective reactions. The 3,5-dimethyl anilide of (−)-proline was acylated on the ring nitrogen with a mixture of t-octanoyl chlorides. The mixture was non-crystalline and was quite soluble in hydrocarbon solvents such as hexane or decane.

Similar mixed selector compositions were prepared from the anilide-like derivatives of (−)-proline made from p-toluidine, aniline, alpha-naphtylamine, and beta-naphtylamine. The latter two were less soluble than the former two and afforded less enantioselectivity during the extraction process. The selectors derived from p-toluidine and from aniline were marginally less enantioselective in the extraction process but were less expensive to prepare. Accordingly, these might be more preferable for large-scale reactions.

The methods and chiral compositions described herein are also contemplated for use in membrane systems that are used to separate enantiomers, particularly in hollow fiber membrane systems. For description of such membrane systems and their use, see, for example, William H. Pirkle and William E. Bowen, Tetrahedron: Assymetry, Vol. 5, No. 5, pp.773-776, 1994, and William H. Pirkle and Elizabeth M. Doherty, Journal of the American Chemical Society, 111, pp. 4113-4114, 1989.

Briefly, membrane systems comprise a source phase, carrier phase, and receiving phase. Permeable membranes are located at interfaces between the source phase and carrier phase and between the carrier phase and receiving phase. The racemate is dissolved in the aqueous source phase, whereas the PTC and chiral selector are dissolved in the organic solvent carrier phase. The enantiomers interact with the PTC, and the preferred enantiomer/PTC complex extracts quickly into the organic carrier phase due to the presence of the chiral selector therein. The complex then travels downstream towards the aqueous receiving phase. While not being bound to a particular theory, it is believed that the complex, due to rapid equilibrium, disrupts prior to crossing the carrier phase/receiving phase interface. As a result, it is mostly the preferred enantiomer that accumulates in the receiving phase, whereas the PTC and selector are free to interact with new enantiomers from the source phase. It should be noted that as with the other enantioselective reactions described herein, the highest enantioselectivity is achieved early in the reaction before the racemate becomes depleted in the source phase. Accordingly, enantioselectivity can be kept high if there is a continuous supply of the racemate in the source phase.

To be of significant commercial value, the chiral selectors used in membrane sytems are preferably relatively inexpensive and afford high levels of enantioselectivity. Furthermore, they are preferably relatively soluble in a solvent, otherwise the precipitation of selector may cause clogging of membrane tubing, thereby rendering such membrane system less effective. Therefore, the use of a mixture of chiral selectors that comprise more than one chiral selector and achieve higher solubility would be particularly useful in membrane systems for enantioselective separation.

In a membrane system as described herein, the increase in capacity of the membrane results in the greater rate of transport of the substrate into the receiving phase. As a result, mixtures of selectors would present a means of greatly improving membrane efficiency with likely improvement in selectivity. Consequently, the increase in concentration of substrate in the organic phase indicates a potentially beneficial result in membrane separations. The efficiency of a membrane would increase with little to no loss of ee.

In an alternative embodiment of the present invention, the membrane system may comprise a source phase and at least two separate carrier phases, wherein the source phase comprises a racemic mixture to be resolved and each of the carrier phases comprises at least two chiral selectors wherein the chiral selectors in different carrier phases preferentially associate with the different enantiomers of the racemic mixture.

In another embodiment, each carrier phase comprises at least two separate chiral selectors that have identical core structures that are responsible for enantioselective chiral recognition but differ in at least one substituent. Furthermore, such membrane system may comprise at least two receiving phases, wherein each of the enantiomers accumulates. For example, an aqueous source phase comprising a racemate to be resolved may be separated by permeable membrane(s) from two or more separate organic solvent carrier phases, each comprising a phase transfer catalyst and at least two chiral selectors selected from the mixture of selectors used to resolve a particular enantiomer. Furthermore, this membrane system may contain at least two receiving phases, separated by membrane(s) from the carrier phases, wherein the resolved enantiomers accumulate.

In one embodiment, at least one carrier phase comprised in the membrane systems described herein comprises at least two proline-derived chiral selectors. In another embodiment, proline-derived selectors comprise (S)-N-butanoylproline-3,5-dimethylanilide (BPA) and (S)-N-pivaloylproline-3,5-dimethylanilide (PPA).

Another feature contemplated within the scope of the present invention is the use of chiral selector mixtures, wherein said mixtures comprise chiral selectors that are poorly soluble but exhibit high chiral recognition. It should be noted that the chiral selectors used in such mixtures comprise the identical “core” structure responsible for specificity of chiral recognition and at least one different substituent attached to said core. By using such compositions, the chiral selectors comprised therein may each be solubilized to a greater degree simply by mixing several poorly soluble selectors together, thus altering the ordered arrangement within the crystal lattice and resulting in improved solubility.

Other features, objects and advantages of the present invention will be apparent to those skilled in the art. The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the present invention.

All publications and patent applications cited in this specification are herein incorporated by reference as if each individual publication or patent application was specifically and individually indicated to be incorporated by reference.

### EXAMPLES

A.) Biphasic Enantioselective Esterification

Using a standard reaction protocol, 1.0 mol equivalent each of racemic DNBleu, 1, and 0.5 mol equivalents of BPB, were stirred in a biphasic solvent system of aqueous sodium bicarbonate and carbon tetrachloride. Little or no esterification occurs at room temperature. In a second reaction, 1.0 mol equivalent of the L-proline derived selector, (S)-2, is added to the above, but does not alter the results. In a third reaction, a small amount of achiral THAC (APTC) is added, without selector (S)-2 present, and leads to the production of racemic ester 3. In a fourth reaction, achiral THAC and (S)-2 are added together to the above, resulting in enantiomerically enriched ester 3. Several examples are given below (Table 1).

TABLE 1
Enantioselective biphasic esterification reactions^{a}

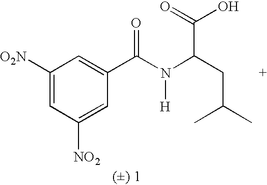

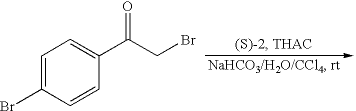

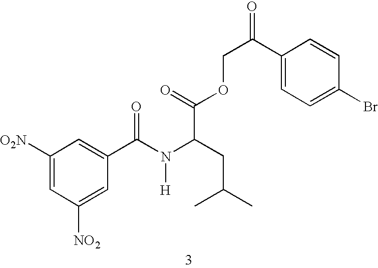

| (±) 1 | BPB | (S)-2 | THAC | % ee | |
|---|---|---|---|---|---|
| (equiv) | (equiv) | (equiv) | (equiv) | (S)-3 | s^{b} |
| 1.0 | 0.50 | 2.00 | 0.035 | 87 | 25.8 |
| 1.0 | 0.50 | 1.00 | 0.035 | 76 | 12.2 |
| 1.0 | 0.50 | 0.50 | 0.035 | 63 | 6.60 |
| 1.0 | 0.50 | 0.25 | 0.035 | 44 | 3.39 |
| 1.0 | 0.50 | 2.00 | 0.085 | 74 | 10.9 |
| 1.0 | 0.50 | 1.00 | 0.085 | 62 | 6.33 |
| ^{a}Standard conditions entailed use of 0.11 mmol (1 mol equiv.) of (±) 1 and the indicated number of mol equiv. of the other reagents in 2.2 mL saturated sodium bicarbonate and 2.2 mL of CCl₄. The reaction was rapidly stirred magnetically at rt. Aliquots were assayed periodically on racemic (extent of conversion) and (R, R)-Whelk-O (% ee) HPLC columns. The selector was | | | | | |
| # utilized as the internal standard. Enantiomeric excess values are reported at the point where 40% of the initially racemic 1 had been converted to ester (i.e. 80% of the theoretical yield). | | | | | |

^{b}Stereoselectivity factor.

B.) Enantioselective Hydrolysis Reactions

When racemic ester 3 is dissolved in an organic solvent and a solution of 5% Na₂CO₃ is added, little or no hydrolysis takes place at room temperature. The addition of an achiral quaternary ammonium salt (e.g. THAC) results in hydrolysis of the ester. When the same reaction is carried out in the presence of selector (S)-2, the hydrolysis reaction occurs in an enantioselective manner, with the residual unreactive enantiomer being enriched in the more complexed enantiomer (Table 2). Acidification of the aqueous layer and subsequent extraction into an organic solvent gives enantiomerically enriched DNBleu (1). The enantioselectivity of the process is generally dependent upon the degree of complexation of the reacting ester. Hence, increasing the concentration of the chiral selector and lowering the temperature results in enhanced enantioselectivity. Applicants also found that decreasing the amount of the achiral quaternary ammonium salt leads to increased enantioselectivity, albeit with decreased rate of hydrolysis.

TABLE 2
Enantioselective Hydrolysis of ±3 in the presence of selector (S)-2
and THAC

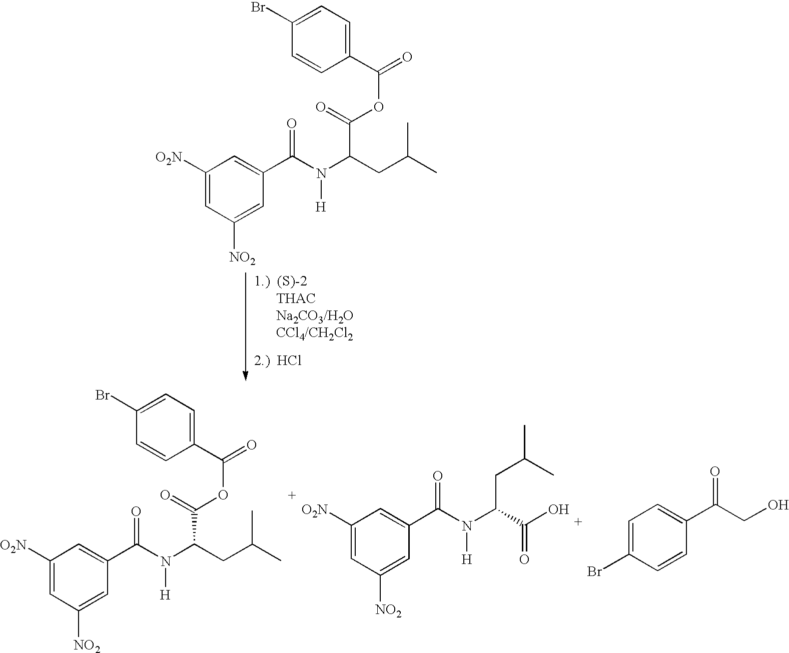

| (±) 3 | (S)-2 | THAC | | % ee | |
|---|---|---|---|---|---|
| (equiv) | (equiv) | (equiv) | TEMP(° C.) | (S)-3 | s^{b} |
| 1.0 | 2.4 | 1.0 | 24 | 20 | 1.8 |
| 1.0 | 2.4 | 0.5 | 24 | 43 | 3.8 |
| 1.0 | 2.4 | 0.2 | 24 | 58 | 6.6 |
| 1.0 | 2.4 | 0.2 | 0 | 78 | 19.0 |
| 1.0 | 1.0 | 0.2 | 24 | 36 | 2.9 |
| ^{a}Standard conditions entailed use of 0.01 mmol (1 mol equiv.) of (±) 3 and the indicated number of mol equiv. of the other reagents in 2.5 mL CCl₄ and 0.5 mL CH₂Cl₂. To this mixture was added 3.0 mL 5% Na₂CO₃. The reaction was rapidly stirred magnetically at the indicated temperature. Aliquots were assayed periodically on racemic (extent of conversion) and | | | | | |
| # (R, R)-Whelk-O (% ee). HPLC columns. The selector was utilized as the internal standard. Enantiomeric excess values are reported at the point where 50% of the initially racemic 3 had been converted. | | | | | |

^{b}Stereoselectivity factor.

It should be pointed out that the methyl and ethyl esters of DNBleu also undergo enantioselective hydrolysis under phase transfer conditions in the presence of a proline-derived selector. In these cases, it is necessary to adjust the pH of the aqueous layer to achieve appreciable rates of hydrolysis. The methyl and ethyl esters are soluble in hexane when the selector is present. Performing reactions in hexane results in increased levels of enantioselectivity. Stereoselectivity factors of 15 and above are commonly achieved running reactions in hexane, with conditions similar to those stated above (see Table 2). Furthermore, although the nature of the chiral selector influences the enantioselectivity of a given hydrolysis reaction, the overall phenomena is quite general and hence, a variety of chiral selectors have been used resulting in similar data.

C.) Enantioselective Nucleophilic Aromatic Substitution

When 4, the racemic amide of DNBleu, is added to a solution of a nonpolar solvent such as carbon tetrachloride or hexane in the presence of chiral selector (S)-2, and a solution of 2M NaOH is subsequently added, no reaction takes place. If the same reaction is run in the presence of THAC or some other achiral quaternary ammonium salt, the organic layer becomes burgundy colored and the amount of 4 in the organic layer is diminished, with the extent of conversion being equivalent to the amount of THAC initially added. Upon acidic workup, regeneration of the original amide is observed. NMR studies suggest that a stable Meisenheimer adduct develops as hydroxide is carried into the organic layer by THAC. Monitoring the residual amide by chiral HPLC shows that the process is enantioselective with the more highly complexed enantiomer protected from reaction. Under certain conditions, particularly when reactions are carried out in hexane, the Meisenheimer adduct precipitates from solution and can subsequently be separated from residual amide dissolved in the organic layer. Acidic workup of the isolated amide precipitate leads to highly enantioenriched amide. The ester remaining in solution is highly enriched in the other enantiomer. Thus, both enantiomers are obtained and the chiral selector can be recovered by conventional means and be reused. Stereoselectivity factors exceeding 25 are commonly obtained when such reactions are run at 0° C.

Similar enantioselective Meisenheimer adducts have been generated using compounds containing electron deficient aromatic moieties. Of particular interest is enantioselective reactions performed on the commercially available racemic Whelk-O selector (6).

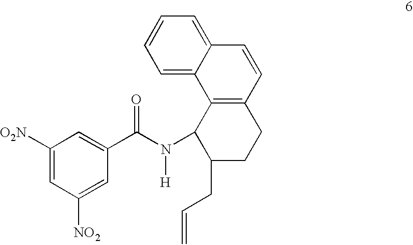

Under a standard set of conditions for reactions run in a biphasic solution of carbon tetrachloride and 2M NaOH at 0° C., using two molar equivalents of selector (S)-2 and a half of an equivalent of THAC per 1 molar equivalent of racemic 6, stereoselectivity factors exceeding 10 can be achieved. Furthermore, the reactions are not limited to using sodium hydroxide as a nucleophile. For instance, enantioselective Meisenheimer adducts can be generated using aqueous solutions of sodium peroxide.

D.) One-pot Enantioselective Esterification/Hydrolysis

Typical procedure for biphasic hydrolysis included: Racemic 1 (0.023 mmol) and (S)-2 (0.046 mmol) were dissolved in 2.9 ml hexane and 0.15 ml of CH₂Cl₂. Solution was stirred rapidly at room temperature and 3.0 ml of 2M sodium hydroxide was added. Reaction progress was monitored periodically by HPLC (the selector, (S)-2, was used as an internal standard). The reaction was stirred for 1 hour, at which point HPLC indicated 50% conversion. The layers were separated and the aqueous layer was extracted three times with CH₂Cl₂. The combined organic layers were dried over MgSO4, filtered, and concentrated under reduced pressure. Separation of 1 and (S)-2 was done by flash column chromatography (SiO₂, hexane/ethyl acetate).

To determine the enantiomeric excess of the N-acylated amino acid in original aqueous layer: 2M HCl was added slowly until pH was approximately 8. To this solution, 0.023 mmol of tetrahexylammonium bromide in 5.0 ml of CH₂Cl₂ was added. Layers were equilibrated by rapid stirring for 10 minutes. The layers were separated and the organic layer was dried over MgSO₄ and concentrated under reduced pressure. Methylation of carboxylate ion-pairs was accomplished by adding 0.023 mmol of dimethyl sulfate dissolved in 1.0 ml of CH₂Cl₂. The solvent was evaporated under reduced pressure and the crude methyl ester was purified by flash column chromatography (SiO₂, hexyl/ethyl acetate). Enantiomeric excess of the methyl ester was determined by using a chiral stationary phase ((R,R)-Whelk 01, (10% iPrOH in hexane)). Enantioselective hydrolysis was tested using a number of racemic esters, which included racemic esters of N-3,5-dinitrobenzoyl amino acids as shown in 1a-e.

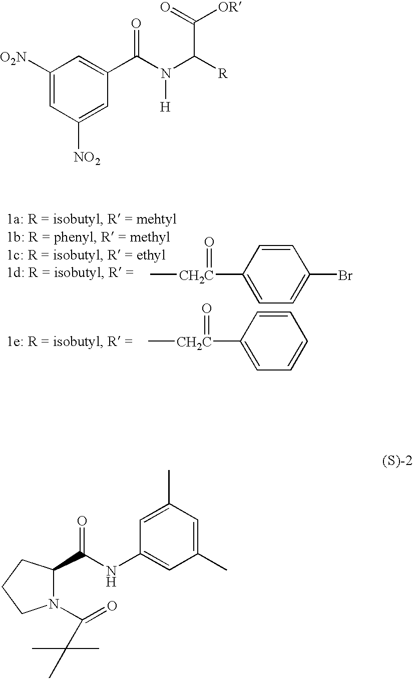

Each racemic ester was stirred in nonpolar organic solvent with 2M sodium hydroxide in the presence of a water immiscible solution of the chiral selector. Selector (S)-2 was used as a chiral complexing agent. Table in Scheme 3 provides several examples of the effects of selector concentration, temperature, and organic solvent on the apparent stereoselectivity factor, s, of hydrolysis. The enantiomeric purities in Table in Scheme 3 are reported for hydrolysis of 50% of the ester initially present, the required times varying between 30 minutes and four hours. Preferential hydrolysis of the less complexed enantiomer is observed in all cases, reaction rate increasing markedly with stirring speed. The reaction occurs at the interface since reaction rate increases with increased interfacial surface area. Hydrolysis also occurs more rapidly in the absence of the selector, demonstrating that complexation inhibits hydrolysis.

Increasing the concentration of the selector slows hydrolysis and increases enantioselectivity. The presence of a phase transfer catalyst is not essential to the hydrolysis but does influence rate.

Once hydrolysis has proceeded to the desired extent, isolation and acidification of the aqueous layer liberated the enantioenriched carboxylic acid. (Carboxylic acid obtained by hydrolysis moves into the aqueous phase if there are no ammonium ions, however in the presence of ammonium ions, it is likely that most but not all of the carboxylic acid migrates into the aqueous layer). The chiral selector and the enantioenriched ester remain in the organic layer and can be recovered either by chromatography on silica or by hydrolysis of the ester and extraction of the enantioenriched acid.

Hydrolysis of the esters was faster (at least twenty times for the exemplary esters) in hexane than in methylene chloride and the enantioselectivity was greater as well.

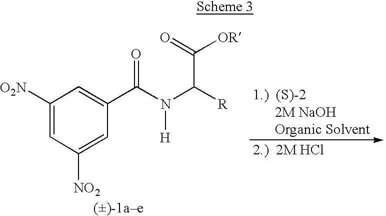

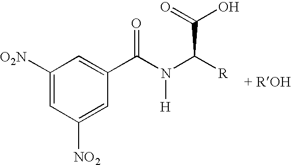

| Entry | Ester | (S)-2 (eq.) | T (° C.) | Solvent | % ee^{[a]} | S^{[f]} |
|---|---|---|---|---|---|---|
| 1 | 1a | 2.0 | rt | Hex/CH₂Cl₂^{[b]} | 83.0 | 27.8 |
| 2 | 1a | 2.0 | 0 | Hex/CH₂Cl₂^{[b]} | 91.5 | 70.2 |
| 3 | 1a | 2.0 | rt | CCl₄/CH₂Cl₂^{[c]} | 79.0 | 20.3 |
| 4 | 1a | 0.5 | rt | Hex/CH₂Cl₂^{[b]} | 59.0 | 6.9 |
| 5 | 1a | 0.5 | 0 | Hex/CH₂Cl₂^{[b]} | 67.0 | 8.5 |
| 6 | 1b | 2.0 | rt | CCl₄/CH₂Cl₂^{[c]} | 61.0 | 7.5 |
| 7 | 1c | 2.0 | rt | Hex/CH₂Cl₂^{[b]} | 86.0 | 36.7 |
| 8 | 1c | 2.0 | 0 | Hex/CH₂Cl₂^{[b]} | 92.0 | 80.0 |
| 9 | 1d | 2.0 | rt | CCl₄/CH₂Cl₂^{[c]} | 65.0 | 9.0 |
| 10 | 1e | 2.0 | rt | CCl₄/CH₂Cl₂^{[c]} | 70.0 | 11.7 |
| 11 | 1e | 2.0 | rt | Hex/CH₂Cl₂^{[b]} | 79.0 | 20.3 |

^{[a]}Standard conditions entailed use of 0.023 mmol (1 mol eq.) of racemic ester and the indicated number of molar eq. (S)-2 in the indicated organic solvent and 3.0 mL of 2M sodium hydroxide. The reaction was rapidly stirred magnetically. Aliquots were assayed periodically by chiral HPLC.
^{[b]}Racemic ester and (S)-2 dissolved in 2.9 mL of hexand and 0.15 mL of CH₂Cl₂.
^{[c]}Racemic ester and (S)-2 dissolved in 2.5 mL of CCL₄ and 0.5 mL of of CH₂Cl₂.
^{[d]}Racemic ester and (S)-2 dissolved in 2.5 mL of hexane and 0.5 mL of CH₂Cl₂.
^{[e]}% ee of both the residual ester (enriched in (S) enantiomer) and the product DNB amino acids (enriched in the (R) enantiomer) at 50% conversion determined by using a chiral stationary phase (1a, 1b and 1d: (R,R)-Whelk O1 (13% iPrOH in hexane) available from Regis Technologies; 1c and 1e: (D)-Leucine (10% iPrOH in hexane) available from Regis Technologies). Absolute configurations were assigned by comparison with authentic samples.
^{[f]}Stereoselectivity factor.

Increasing the pH of the aqueous solution once the esterification reaction is complete improves the rate of hydrolysis reaction. The interplay between concentration of various components, reaction times, yields, and enantiomeric purities is complex. The examples chosen show the power of the tandem process even when the initial alkylation proceeds with modest enantioselectivity. In one example, a solution of 0.05 mmol of phenacyl bromide, 0.003 mmol of tetrahexylammonium bromide (THAB) and 0.10 mmol of (S)-2 in 2.75 ml of carbon tetrachloride and 0.10 ml of dichloromethane was stirred with a solution of 0.10 mmol racemic N-3,5-dinitrobenzoyl leucine in 1.25 ml saturated sodium bicarbonate solution for 27 hours. HPLC analysis showed the organic layer to contain 0.0496 mole of (S)-ester of 70% ee. Addition of 1.25 ml 2M sodium hydroxide to the stirred mixture reduced the amount of ester present in the organic layer to 0.033 mmol with an ee of 100% after four hours. The residual (R)-acid isolated from the aqueous layer was found to be of 55% ee. Increasing the amount of phenacyl bromide to 0.065 mmol led to 0.064 mmol of ester of 49% ee and the subsequent hydrolysis was stopped at the point where 0.040 mmol of (S)-ester of 99% ee remained. The recovered (R)-acid was of 62% ee.

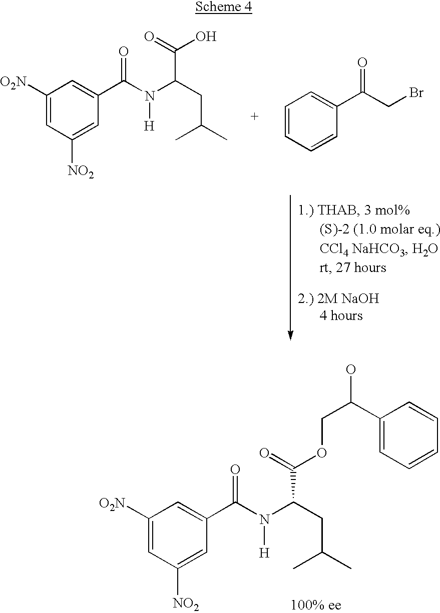

E.) Formation of Meisenheimer Complexes

Racemic DNB amides (1a-d) and achiral amide 1e were chosen as substrates.

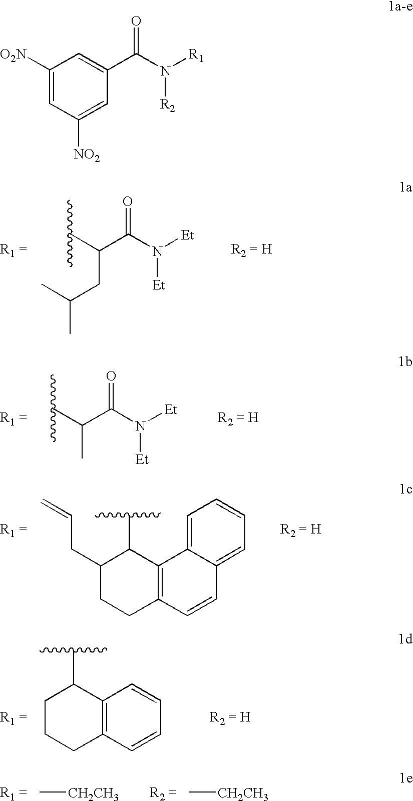

Selectors S-3 and R-4, each of which show a high affinity for π-acidic compounds such as 1a-d were used as chiral complexing agents.

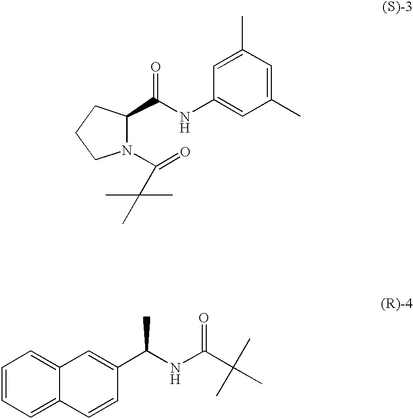

As shown in Table 4, substantial enantioselectivities are usually achieved and in the case of (±) 1a, the stereoselectivity factor approached the maximum value that can be experimentally determined for a kinetic resolution (s>100).

TABLE 4
Generation of Enantioselective Meisenheimer Complexes^{a}

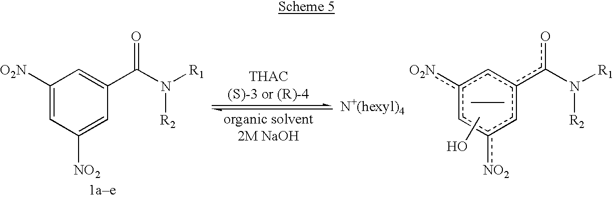

| Entry | Amide | Selector | Solvent | T(° C.) | % conv.^{b} | % ee | s^{c} |
|---|---|---|---|---|---|---|---|
| 1 | (±) 1a | (S)-3 | CCl₄ | 22 | 50 | 85 (S) | 33.0 |
| 2 | (±) 1a | (S)-3 | Hexane | 22 | 30 | 42 (S) | >100 |
| 3 | (±) 1b | (S)-3 | CCl₄ | 22 | 50 | 65 (S) | 9.0 |
| 4 | (±) 1b | (S)-3 | Hexane | 22 | 41 | 50 (S) | 9.5 |
| 5 | (±) 1b | (S)-3 | Hexane | 0 | 40 | 58 (S) | 26.0 |
| 6 | (±) 1c | (S)-3 | CCl₄ | 22 | 46 | 53 (S) | 7.0 |
| 7 | (±) 1c | (S)-3 | Hexane | 22 | 43 | 51 (S) | 8.0 |
| 8 | (±) 1c | (S)-3 | Hexane | 0 | 39 | 50 (S) | 13.5 |
| 9 | (±) 1c | (R)-3 | CCl₄ | 22 | 50 | 70 (R) | 11.5 |
| 10 | (±) 1c | (R)-4 | CCl₄ | 0 | 41 | 58 (R) | 18.0 |
| 11 | (±) 1d | (R)-4 | Hexane | 0 | 44 | 20 (R) | 2.0 |

^{a}Standard conditions entailed use of 0.01 mmol (1 mol equiv.) of racemic amide, 0.02 mmol (2 mol equiv.) of selector, and 0.005 mmol (0.5 mol equiv.) of THAC added to 2.0 mL of the indicated solvent, 0.1 mL of methylene chloride, and 2 mL of 2M NaOH. The reaction was rapidly stirred magentically. Aliquota were assayed periodically by chiral HPLC.
^{b}Conversion based on residual unreacted amide.
^{c}Stereoselectivity factor.

For reactions run in CCl₄ at room temperature, with one-half molar alent of THAC added for each molar equivalent of 1a-e, approximately 5% of 1a-e is consumed within the first few minutes. The extent of reaction ultimately reaches the molar quantity of THAC present after approximately 24 hours. The organic layer remains a homogenous burgundy color throughout the course of the reaction. The presence of one molar equivalent of acetone in an otherwise identical experiment leads to the formation of a dark blue color, the consumption of amide approaching 50% within minutes. These observations can be explained through the formation of a different, more thermodynamically stable MC. Ambident acetonate anions attach aromatic nitro compounds to generate Meisneheimer complexes, the C-adducts being more stable than the corresponding O-adducts. The enantioselectivity of C-adduct formation is comparable to that of the O-adduct formed from reaction with hydroxide ions.

Treatment of these complexes with acid leads to regeneration of 1a-e as well as to products resulting from oxidation of the MC. The extent of oxidation is heavily dependent upon reaction times and temperature and whether oxygen has been excluded from the reaction. Reaction of the achiral amide 1e in CCl₄ with 2M NaOH in the presence of THAC results in a loss of approximately 25% of the amide to oxidative products upon acidic workup. The ipso-substituted product resulting from displacement of a nitro group by a hydroxyl group accounts for the majority of the recovered oxidation products. With acetenoate as a nucleophile, oxidation of the C-adduct at the ortho-position leads to the Janovsky product. Similar oxidative products are generated with the chiral amide 1c. In the presence of a chiral complexing agent, the recovered Janovsky products are enriched in the enantiomer derived from the less complexed enantiomer of the amide. Reactions carried out under oxygen-free conditions give increased levels of amide regeneration upon acidification. The small quantity of the MC that is oxidized may arise through intermolecular self-oxidation by “spontaneous” aromatization.

For reactions in acetone-free hexane, reaction rates are increased markedly and the amount of oxidation products formed are negligible owing to the phase separation of 2 as a highly viscous burgundy colored sludge-like salt. Dissolution of this sludge in methylene chloride, followed by acidification, results in regeneration of the amide enantiomer having the opposite configuration of the enantiomer that remains in the hexane. In this sludge, the ratio of the quaternary ammonium cation of THAC to the anionic MC is generally greater than one, although the exact ratio is case-dependent. The unique properties of the Meisenheimer complexes in these biphasic systems are attributed to the formation of a stable ion-pair between the cation of THAC and the negatively charged MC. Interestingly, the stabilized ion-pairs (2) show properties similar to Meisenheimer adducts generated in water-dimethyl sulphoxide (DMSO) mixtures. Ultraviolet/Visible spectra of the ion-pairs of 1e generated in CCl4 closely resemble Cramton's data for a similar compound taken in water-DMSO. Furthermore, NMR experiments in d₆-benzene/D2O demonstrate that there is extensive hydrogen exchange of aromatic protons in the MC generated from 1c and 1e and considerable line broadening is noted for the signals associated with the unreacted amide. Sharp signals for the MC are observed, consistent with previously reported data. The line broadening observed in NMR studies implicates the formation of a radical, a result confirmed by EPR data.

F.) Enantioselective Aromatic Substitution

Biphasic approach has also been tested in enantioselective aromatic substitution reaction, which is known to proceed through the formation of a MC. A p-chloro substituted DNB amide (±5) dissolved in CCl₄ was exposed to a NaOH/methanol solution in the presence of selector R-4 and THAC to afford the enantioenriched methoxy substituted product (6) (Scheme 6).

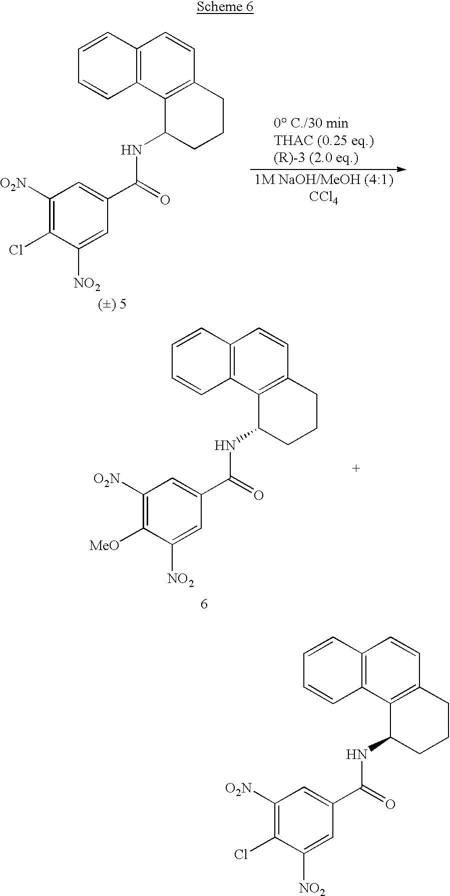

When the reaction was assayed after 10 minutes, at which point 50% conversion of (±5) had been achieved, the ee% of both the residual substrate and product were approximately 60% (at 72% conversion, the ee% of residual 5 was 96%).

G.) Use of Mixtures of Chiral Selectors

The chiral selector that was used was (S)-N-acylproline-3,5-dimethylanilide, I. This selector is highly hydrophobic. Additionally, the chiral center contained in the proline subunit is less susceptible to loss of chiral integrity, as compared with other amino acids. This selector was developed for an LC system, where it exhibited a high level of recognition for N-derivatized amino acids. A recognition mechanism was proposed utilizing N-3,5-dinitrobenzoylleucine as the representative racemate. The mechanism was supported utilizing solution state NMR evidence and x-ray crystallographic data. As a result, the chiral recognition for this selector and racemate is well understood.

The substrate, (R,S)-3,5-dinitrobenzoylleucine was used in our studies due to I's high affinity for a single enantiomer of the racemate. Whenever a proline-based chiral selector is used, the substrate has the added requirement of containing an acidic proton at the carboxylic oxygen. In basic solution, this proton is easily removed providing the necessary anionic site for quat interaction.

Saturated mixtures of the chiral selectors, (S)-N-butanoylproline-3,5-dimethylanilide (BPA) and (S)-N-pivaloylproline-3,5-dimethylanilide (PPA), as well as saturated solutions containing the selectors individually were prepared and used in this experiment.

Depicted below are the two chiral selectors and the racemic substrate, (R,S)-3,5-dinitrobenzoylleucine (DNB leucine) that were used in this experiment.

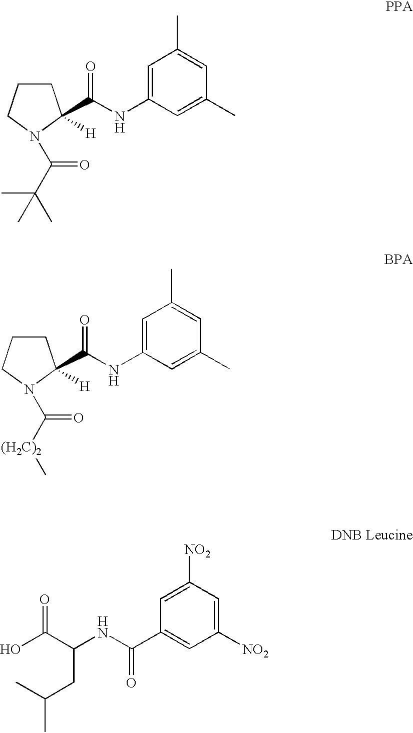

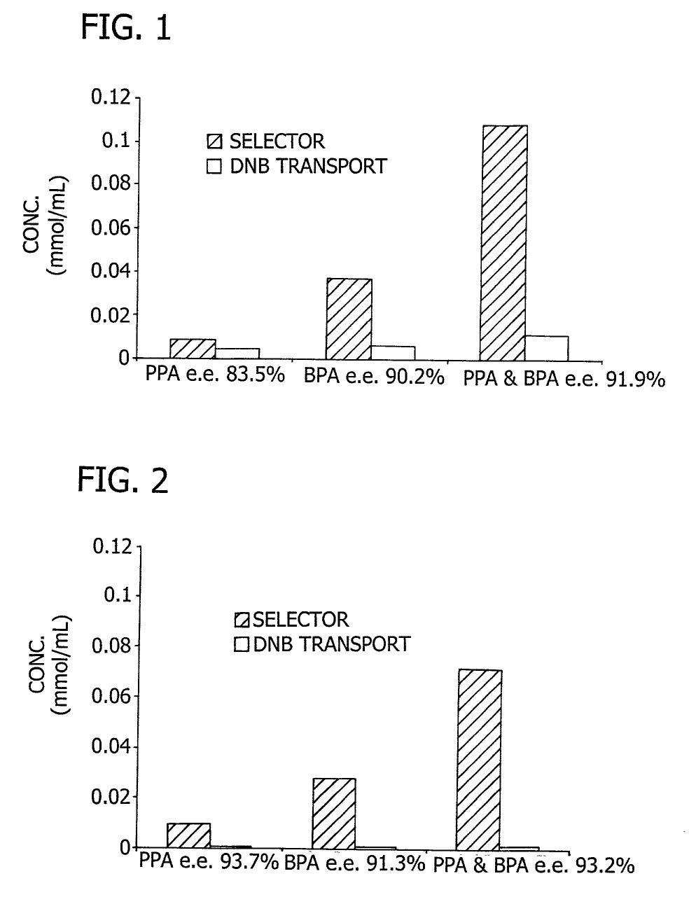

FIGS. 1 and 2 present the results of studies in which saturated mixtures of BPA and PPA are compared to the performance of individual saturated selectors in biphasic extraction. Each of the extractions was performed utilizing the same biphasic composition, the same quat concentration, wherein the quat was tetrahexyl ammonium chloride in FIG. 1 and tetrabutyl ammonium bromide in FIG. 2, and the same substrate concentration, wherein the substrate was (R,S)-3,5-dinitrobenzoylleucine.

The results demonstrate that mixing selectors allows for an increase in overall concentration of the chiral selector. Apparently, the selectors act as impurities toward one another affecting solid formation by disrupting the formation of crystals. This method of increasing overall selector concentration does not introduce any extraneous interactions that other additives may introduce. Extraneous interactions would detrimentally affect the chiral recognition mechanism, adversely affecting the overall extraction. Importantly, the mixtures of selectors out-perform the single selectors by themselves due to the increased selector concentration in the mixtures.

The mixtures in both studies allowed for the transfer of over twice the amount of substrate as compared with the single selectors while maintaining high ee levels.

It is further demonstrated that quat's alkyl chain length has an effect on substrate that's transferred as well as on selectivity of the transfer. When optimizing a separation, the optimal balance between selectivity and concentration of substrate transferred can easily be adjusted to optimize the separation. As is demonstrated by these studies, the tetrahexyl quat achieves higher substrate transport while the tetrabutyl quat achieves higher ee values.

An important variable in these extractions is the relative concentration of substrate transferred and the relative concentration of selector present. This is especially evident when the relative concentration of PPA to substrate in FIG. 1 is approximately 2:1 and the ee value is 83.5%. When the relative concentration in FIG. 2 for the PPA separation is 4:1, the ee value jumps to 93.7%. The PPA selector in FIG. 1 appears to be a poorer selector simply because it is not sufficiently concentrated to perform optimally. But, as the relative amount of selector to substrate increases, the selector performs better, demonstrating that it is the best selector used in the study. It was also determined that the different substituents on chiral selectors effect the extraction process to a small extent. Likely, the bulky substituent of PPA holds the selector in a conformation more conducive to selective enantiomer extraction. The conformational rigidity may also help to explain the poor solubility of PPA. Nevertheless, the results demonstrate improvement with the mixture of selectors.

Materials and Methods

All syntheses were performed under nitrogen utilizing standard glassware. Parapbromophenacyl bromide (Aldrich), cyclohexane (Fisher), NaHCO3 (Fisher), tetrahexyl ammonium chloride (Aldrich), tetrabutyl ammonium bromide (Aldrich), palmitic acid (Aldrich), thionyl chloride (Aeros), 2-propanol (Fisher), DL-leucine (Aldrich), 3,5-dinitrobenzoyl chloride (Aldrich), tetrahydrofuran (Fisher), propylene oxide (Aldrich), acetone (Fisher), CCl₄ (Fisher), butanoic acid (Adrich), L Proline (Aldrich), 3,5-dimethylaniline (Aldrich), NaOH pellets (fisher), diethyl ether (Fisher), concentrated HCl (Fisher), anhydrous MgSO₄ (Fisher), and triethylamine (Aldrich) were used without further purification. Hexane, ethyl acetate, and CH₂Cl₂ used in extractions and chromatography were distilled. Dry CH₂Cl₂ used in synthesis was distilled under CaH₂. EEDQ (Aldrich) was crystallized from hexane proper to use.

Racemic dinitrobenzoyl leucine. DL-leucine (2.00 g, 15.2 mmol) and dinitrobenzoyl chloride (3.80 g, 16.7 mmol) were suspended in tetra hydrofuran (100 ml). Propylene oxide (1.00 g, 17.2 mmol, ml) was subsequently added dropwise to the suspension over 5 minutes. The solution gradually turned light yellow-brown as it stirred for the following 2 hours. The solvent was then removed by rotary evaporation. The remaining yellow-brown solid was recrystalized from acetone-CCl₄ producing off-white crystals. Yield was 4.09 g (75%).

Racemic 3,5-dinitrobenzoyllecine-4-bromobenzoate, 2: 1(0.50 g, 1.54 mmol) was ultrasonicated for 15 minutes in aqueous saturated NaHCO₃ (20 ml). Parabromophenacyl bromide (0.47 g, 1.69 mmol) and tetrahexyl ammonium chloride (0.60 g, 1.54 mmol) were dissolved in CH₂Cl₂, this solution was combined with the aqueous solution and stirred 15 minutes. The organic phase quickly turned deep purple. The organic layer was separated and washed with aqueous saturated NaHCO₃ (3×20 ml). The remaining organic solvent was evaporated by rotary evaporation to remove the remaining solvent, leaving a metallic purple oil. The product was purified by silica chromatography using a 1:1 hexane:ethyl acetate eluate, removing the remaining ion pairs. The resulting light brown fractions were combined and evaporated by rotary evaporation. The yellow-brown solid was recrystallized from 1:1 hexane: CH₂Cl₂ resulting in a white powder, which was collected on a glass frit and then washed with 1:1 hexane: CH₂Cl₂ (2×30 ml). Yield was 0.24 g (30%). The mixture was determined to be racemic by HPLC.

Butanoyl chloride 3: Butanoic acid (7.03 g, 0.0798 mol, 7.30 ml) was refluxed with excess thionyl chloride (32.62 g, 0.274 mol, 20.0 ml) for two hours producing a yellow solution. The remaining thionyl chloride was fractionally distilled off. The crude butanoyl chloride was then collected as the second fraction distilled at 101-102° C. producing a clear liquid. Yield was 6.23 g (73.3%).

(S)-N-butanoylproline 4: NaOH pellets (4.4 g, 0.11 mol) were dissolved in water (50 ml) while stirred in an ice bath. (S)-proline (6.34 g, 55.1 mmol) was added to the solution and it was allowed to cool for 15 minutes. 3 (5.34 g, 50.1 mmol, 5.20 ml) was added dropwise over 45 minutes while the solution was stirred in an ice bath. The solution was allowed to mix for an additional hour and then the ice bath was removed and the solution was stirred an additional hour. The aqueous solution was washed with diethyl ether (3×20 ml), and then slowly acidified to pH=1. The resulting solution turned opaque white and was extracted with methylene chloride (4×20 ml). The organic phase was dried over anhydrous magnesium sulfate, and the solvent was removed by rotary evaporation. Finally, the resulting opaque oil was left to dry under vacuum for 8 hours. Yield was 6.16 g (66.4%).

(S)-N-butanoylproline-3,5-dimethylanilide 5: 4 (6.16 g, 33.2 mmol) was dissolved in CH2Cl2 (40 ml). EEDQ (8.21 g, 33.2 mmol) was added to the mixture followed by 3,5-dimethylaniline (4.03 g, 4.14 ml, 33.2 mmol) the solution turned a deep golden color, which was allowed to stir for 12 hours. The solution was rotary evaporated leaving a yellow-brown oil. The oil was dilluted with ethyl acetate (50 ml). The organic layer was washed with 1N H₂SO₄ (3×20 ml) followed by 2N NaOH (2×20 ml). The resulting product was crystallized from methylene chloride and hexane producing a white solid. Yield was 7.84 g (82%).

(S)-N-pivaloylproline-3,5-dimethylanilide 6: 6 was prepared according to the procedure outlined for the synthesis of 5.

Palmitoyl-3,5-dimethylanilide 7: Palmitic acid (10.0 g, 35.1 mmol) was refluxed in SOCl₂ for 2 hours. The remaining SOCl₂ was distilled off. The reaction mixture was then left under vacuum for 15 minutes. The product was added dropwise over the following 30 minutes to a solution of trietylamine (30 ml), CH₂Cl₂ (20 ml), and 3,5-dimethyl aniline (4.25 g, 4.38 ml, 35.1 mmol) cooled in an ice bath. The reaction was allowed to stir for the following 30 minutes. Then the ice bath was removed and the reaction stirred for the following 60 minutes. A white solid formed, which was collected on a glass frit. The solid was washed with NaHCO₃ (3×50 ml). The solid was then dried overnight under vacuum and then recrystallized from hexane 3×'s. Yield was 7.88 g (62.1%).

Enantiomeric Extraction: The enantiomeric extractions were performed by combining cyclohexane (0.95 ml) containing saturated chiral selector, CH₂Cl₂ (0.05 ml) containing the quaternary ammonium cation (0.014 mmol/ml), followed by the aqueous saturated NaHCO₃ solution (1.0 ml) containing 1 (0.07 mmol/ml). The mixture was then stirred for 1 minute and allowed to equilibrate for 15 minutes. An aliquot of the organic layer was then removed (0.5 ml). This aliquot was mixed with an excess of derivatizing agent, parabromophenacyl bromide, and an internal standard, hexadecanoyl-(3,5)-dimethylanilide, for at least 24 hours. The resulting mixture was analyzed by HPLC.

Saturation of Chiral Selectors: The saturated solution containing chiral selector was prepared by dissolving the chiral selector(s) in CH₂Cl₂ (2.00 ml) in a 20 ml scintillation vial followed by ultrasonication for 10 minutes. The solvent was then evaporated by house vacuum for 4 hours followed by vacuum for >12 hours. The dry selector was then dissolved in cyclohexane 5 ml and then ultrasonicated for 10 minutes. The solutions were left to stand for 1 week and then used.

HPLC analysis: LC analysis of enantiomeric extractions was performed with an Anspec HPLC Pump and an LDC Analytical UV Monitor Fixed Wavelength Detector (254 nm). Integration of peak areas was obtained with an HP 3394A Integrator. The chiral column used was an R,R-Whelko column commercially available from Regis technologies. The mobile phase used for the analysis was 15% 2-propanol and 85% hexane. The flow rate was 2.0 ml/min.

In light of the detailed description of the invention and the examples presented above, it can be appreciated that the several aspects of the invention are achieved.

What is claimed is:

1. A method for converting a racemic mixture to an asymmetric product in a biphasic solvent system, said method comprising: forming a solution which includes a pair of enantiomeric ions, a chiral selector, an achiral phase transfer catalyst, and aqueous and organic solvents, wherein the chiral selector enantioselectively complexes with a first enantiomer of the racemic ion pairs formed from the racemic ions and the phase transfer catalyst and wherein the complexation results in preferential transport of the complexed ion pair from a first liquid phase into a second liquid phase where the complexed ion pair reacts with a pre-selected compound, thereby effecting asymmetric formation of a product derived from the first and preferentially complexed enantiomer.

2. A method comprising: forming a solution containing a racemic compound, a solvent, a chiral selector and a phase transfer catalyst, treating the solution with an immiscible second phase containing a pre-selected reagent which is transported into the liquid phase containing the racemic compound and the chiral selector by the action of the phase transfer catalyst, whereby enantioselective binding of the chiral selector to a first enantiomer of the racemic compound over a second enantiomer causes the enantiomers to differ in reactivity toward the transported pre-selected reagent, resulting in an enantioselective reaction with the transported reagent.

3. A method comprising: forming a solution containing an achiral compound, a solvent, a chiral selector and a phase transfer catalyst; treating the solution with an immiscible second phase containing a pre-selected reagent which is transported into the liquid phase containing the achiral compound and the chiral selector by the action of the phase transfer catalyst, whereby a reaction occurs between the transported pre-selected reagent and the achiral compound in a chiral environment, resulting in an induction in asymmetry.

4. A method for preferential formation of a product of a first enantiomer in a mixture containing a pair of enantiomers of a chiral molecule, the method comprising: forming a mixture containing a first and a second enantiomer of a chiral molecule, a chiral selector, an achiral phase transfer catalyst and aqueous and organic solvents, wherein the chiral selector enantioselectively complexes with the first enantiomer of enantiomeric ion pairs formed from enantiomeric ions and the achiral phase transfer catalyst, and wherein the complexation results in preferential transport of the complexed ion pair from a first liquid phase into a second liquid phase where the complexed ion pair reacts with a pre-selected compound, thereby effecting preferential formation of a product derived from the first and preferentially complexed enantiomer.

5. A method for resolving an enantiomeric mixture, said method comprising forming a multiphase system which comprises an aqueous phase, an organic solvent phase, a phase transfer catalyst, an enantiomeric mixture present in the aqueous phase or the organic solvent phase, a species which is reactive with at least one member of the enantiomeric mixture, and a chiral selector present in the organic phase, the chiral selector preferentially associating with one member of the enantiomeric mixture,
allowing the reactive species to react with the members of the enantiomeric mixture to form a reaction product wherein the rate of accumulation of reaction product dervied from the member of the enantiomeric mixture which preferentially associates with the chiral selector is initially less than the rate of accumulation of reaction product derived from the other member of the enantiomeric mixture, and
separating the reaction product from the member of the enantiomeric mixture which preferentially associates with the chiral selector.

6. The method of any one of claims 1, 2, 3, 4, or 5 wherein the racemic mixture comprises a racemic mixture of an N-acylated amino acid.

7. The method of claim 6 wherein the racemic mixture comprises a racemic mixture of a DNB-derived amino acid.

8. The method of any one of claims 1, 2, 3, 4, or 5 wherein the chiral selector comprises N-acylated L-proline anilide (S)-2.

9. The method of any one of claims 1, 2, 3, 4, or 5 wherein the phase transfer catalyst comprises tetra n-hexyl ammonium halide.

10. The method of claim 9 wherein the tetra n-hexyl ammonium halide is tetra n-hexyl ammonium chloride (THAC).

11. A method for enantioselectively forming a Meisenheimer type adduct from a racemic compound containing an electron deficient aromatic substituent comprising: forming a reaction mixture which includes a pair of enantiomers of a racemic compound containing an electron deficient aromatic substituent, a chiral selector, an achiral PTC, a nucleophile and aqueous and organic solvents; whereby the formation of the solution results in preferential formation of a Meisenheimer type adduct between the nucleophile and a first enantiomer of the pair of enantiomers.

12. The method of claim 11 further comprising separating the adduct from residual substrate resulting in enantioenriched material from a first fraction comprising the residual substrate and a second fraction comprising a substrate recovered from the adduct.

13. The method of claim 11 wherein the reaction mixture comprises a biphasic system comprising an aqueous solution and a nonpolar organic solvent.

14. The method of claim 11 wherein the reaction mixture comprises a triphasic system comprised of an aqueous solution, a nonpolar organic solvent, and the achiral phase transfer catalyst affixed to a solid or liquid polymer.

15. The method of any one of claims 11-14, wherein the chiral selector comprises N-acylated L-proline anilide (S)-2.

16. The method of any one of claims 11-14, wherein the phase transfer catalyst comprises tetra n-hexyl ammonium chloride (THAC).

17. The method of any one of claims 11-14 wherein the nucleophile is selected from the group consisting of a hydroxide ion, a hydroperoxide ion and a mercaptide ion.

18. The method of any one of claims 11-14 wherein the organic solvent is a hydrocarbon or a halocarbon.

19. A mixture of chiral selectors comprising at least two separate chiral selectors that have identical core structures that are responsible for enantioselective chiral recognition but differ in at least one substituent.

20. The mixture of chiral selectors of claim 19 comprising two chiral selectors that have identical core structures and at least one different substituent.

21. The composition of claim 20, wherein the chiral selectors are proline-derived.

22. The composition of claim 21, wherein the chiral selectors comprise (S)-N-butanoylproline-3,5-dimethylanilide (BPA) and (S)-N-pivaloylproline-3,5-dimethylanilide (PPA).

23. The method of claim 1 wherein the biphasic solvent system comprises at least two separate chiral selectors.

24. The method of claim 2 wherein the solution comprises at least two separate chiral selectors.

25. The method of claim 3 wherein the solution comprises at least two separate chiral selectors.

26. The method of claim 4 wherein the mixture comprises at least two separate chiral selectors.

27. The method of claim 5 wherein the multiphase system comprises at least two separate chiral selectors in the organic phase.

28. The method of claim 11 wherein the reaction mixture comprises at least two separate chiral selectors.

29. The method of any one of claims 23-28 wherein said chiral selectors are proline-derived.

30. The method of claim 29, wherein the chiral selectors comprise (S)-N-butanoylproline-3,5-dimethylanilide (BPA) and (S)-N-pivaloylproline-3,5-dimethylanilide (PPA).

31. The method of claim 30 wherein the phase transfer catalyst comprises tetra n-hexyl ammonium halide.

32. The method of claim 31, wherein the tetra n-hexyl ammonium halide is tetra n-hexyl ammonium chloride (THAC).

33. A membrane system used to resolve enantiomers of a racemic mixture, comprising a source phase and a carrier phase, wherein the source phase comprises a racemic mixture to be resolved and the carrier phase comprises the mixture of chiral selectors of claim 19.

34. The membrane system of claim 33 further comprising a receiving phase wherein the preferred enantiomer of said racemic mixture accumulates.

35. The membrane system of claim 33, wherein the mixture of chiral selectors comprises at least two proline-derived chiral selectors.

36. The membrane system of claim 35, wherein said proline-derived chiral selectors comprise (S)-N-butanoylproline-3,5-dimethylanilide (BPA) and (S)-N-pivaloylproline-3,5-dimethylanilide (PPA).

37. A membrane system used to resolve enantiomers of a racemic mixture, comprising a source phase and at least two separate carrier phases, wherein the source phase comprises a racemic mixture to be resolved and each of the carrier phases comprises at least two chiral selectors wherein the chiral selectors in different carrier phases preferentially associate with the different enantiomers of the racemic mixture.

38. The membrane system of claim 37 further comprising at least two receiving phases wherein each of the enantiomers of said racemic mixture accumulates.

39. The membrane system of claim 37 wherein each carrier phase comprises at least two separated chiral selectors that have identical core structures that are responsible for enantionselective chiral recognition but differ in at least one substituent.

40. The membrane system of claim 39, wherein each carrier phase comprises at least two proline-derived chiral selectors.

41. The membrane system of claim 40, wherein said proline-derived chiral selectors comprise (S)-N-butanoylproline-3,5-dimethylanilide (BPA) and (S)-N-pivaloylproline-3,5-dimethylanilide (PPA).
